(19) United States
(12) Patent Application Publication
(10) Pub. No.: US 2018/0318372 A1
(43) Pub. Date: Nov. 8, 2018

(54) Method for Production of an Antimalarial Compound

(71) Applicants: Lawal Bilbis, Sokoto (NG); Rabiu Umar Aliyu, Sokoto (NG); Suleman Y. Mudi, Kano (NG); Yusuf Saidu, Sokoto (NG); Andrew Onu, Sokoto (NG)

(72) Inventors: Lawal Bilbis, Sokoto (NG); Rabiu Umar Aliyu, Sokoto (NG); Suleman Y. Mudi, Kano (NG); Yusuf Saidu, Sokoto (NG); Andrew Onu, Sokoto (NG)

(21) Appl. No.: 15/970,624

(22) Filed: May 3, 2018

Related U.S. Application Data
(60) Provisional application No. 62/500,917, filed on May 3, 2017.

(51) Int. Cl. A61K 36/482; A61P 33/06; A61K 31/498

(52) U.S. Cl. CPC A61K 36/482; A61P 33/06; A61K 2236/51; A61K 2236/331; A61K 2236/39; A61K 31/498

(57) ABSTRACT

A method for the production of an antimalarial compound from a quantity of plant matter obtained from the plant species Cassia Nigricans. Active ingredients are extracted from the quantity of plant matter through infusing the active ingredients into an aqueous solution. The aqueous solution is lyophilized to concentrate the active ingredients. The active ingredients are then separated through a series of chromatography separation processes to produce the antimalarial compound. The antimalarial compound is effective in the treatment of malarial infections.

The current application claims a priority to the U.S. Provisional Patent application Ser. No. 62/500,917 filed on May 3, 2017.

### FIELD OF THE INVENTION

The present invention relates generally to the production for an antimalarial compound. More specifically, the present invention relates to the isolation of a chemical compound effective in the treatment of malarial infection from the plant Cassia nigricans.

### BACKGROUND OF THE INVENTION

Malaria remains the most significant parasitic disease in the tropics where it causes approximately 200 million clinical cases and is reported to claim up to 1.2 million lives each year. To put this in perspective, malaria disease affects the productivity of individuals, families, and the society of a country as a whole. A dramatic recrudescence of malaria is ongoing, and this may not be unconnected to the increasing resistance of mosquito vectors to insecticides and resistance of the parasites, mainly Plasmodium falciparum, to available modern drugs.

Treatment of malaria infection is complicated by resistance of the parasites to most drugs deployed except the more expensive and less available ones like artemisinin-based drugs. Recent reports indicate resistance emerging to artemisinin when used as monotherapy. Resistance gives rise to more severe forms of the disease and spread to malaria-free areas. Therefore, the search of new antimalarial agents must be a continuous process.

Therefore, an objective of the present invention is to provide an antimalarial compound and method of production which includes a chemical compound, that is isolated and characterized from a plant, and is effective in the treatment of malarial infection. The present invention is a method for the production of an antimalarial compound. The antimalarial compound is isolated and characterized from the plant may be modified to enhance its antimalarial activity and improve its safety profile.

### BRIEF DESCRIPTION OF THE DRAWINGS

FIG. 1 is a flow diagram detailing the general process for the present invention.

FIG. 2 is a flow diagram further specifying the present invention.

FIG. 3 is a flow diagram detailing a first alternate embodiment of the present invention, wherein a derivative antimalarial compound is synthesized.

FIG. 4 is a flow diagram detailing a second alternate embodiment of the present invention, wherein a derivative antimalarial compound is synthesized.

FIG. 5 is an ingredient list for the extraction agents of the present invention.

FIG. 6 is an ingredient list for the elution agents of the present invention.

FIG. 7 is an ingredient list for the purification solvents of the present invention.

FIG. 8 is a diagram for the chemical reaction for producing the derivative antimalarial compound from the antimalarial compound for the first alternate embodiment of the present invention.

FIG. 9 is a diagram for the chemical reaction for producing the derivative antimalarial compound from the antimalarial compound for the second alternate embodiment of the present invention.

### DETAIL DESCRIPTIONS OF THE INVENTION

All illustrations of the drawings are for the purpose of describing selected versions of the present invention and are not intended to limit the scope of the present invention.

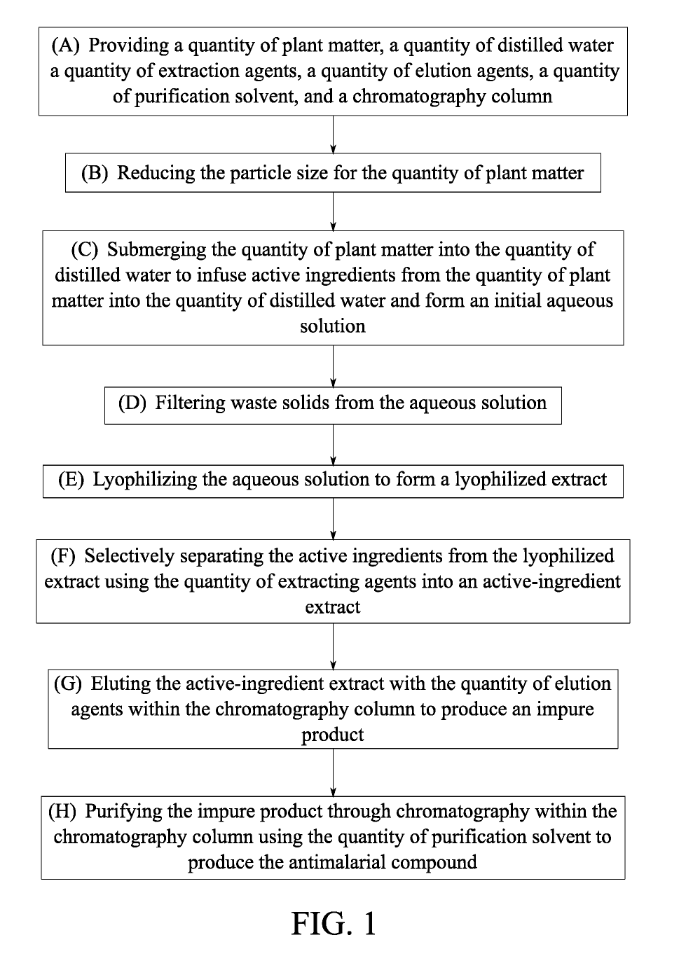

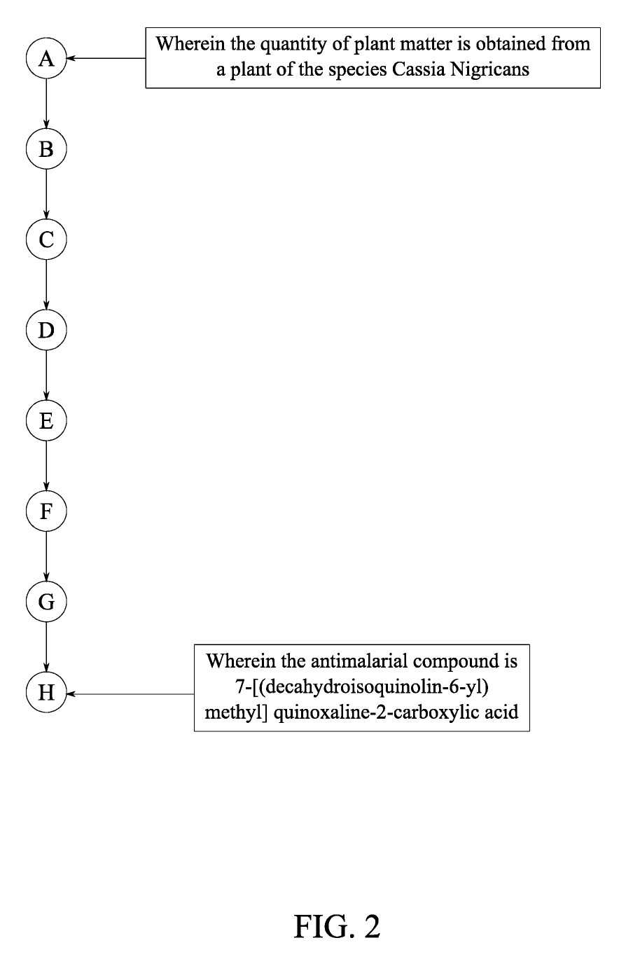

The present invention is a method for the production of an antimalarial compound. The antimalarial compound is isolated from a plant to be effective in the treatment of malarial infection. The present invention requires a quantity of plant matter, a quantity of distilled water, a quantity of extraction agents, a quantity of elution agents, a quantity of purification solvent, and a chromatography column in order to be executed (Step A), shown in FIG. 1. The quantity of plant matter is obtained from stems and leaves from a plant of the species Cassia Nigricans, in accordance to FIG. 2. The Cassia Nigricans species contains an active ingredient that is able to be processed into an antimalarial compound, 7-[(decahydroisoquinolin-6-yl) methyl] quinoxaline-2-carboxylic acid, a desired product to treat malaria. The quantity of distilled water, the quantity of extraction agents, the quantity of elution agents, the quantity of purification solvent, and the chromatography column are utilized in processing the quantity of plant matter into the antimalarial compound.

For the initial step of the present invention, the particle size for the quantity of plant matter is reduced (Step B), further in accordance to FIG. 1. The particle size for the quantity of plant matter is preferred to be reduced through grinding, crushing, or milling or any other appropriate method for granulating plant matter. Reduction of the particle size increases for the quantity of plant matter the surface area and exposes the internal components of the plant matter to allow for the efficient and effective extraction of relevant chemical compounds. The quantity of plant is then submerged into the quantity of distilled water to infuse the active ingredients from the quantity of plant matter into the quantity of distilled water to form an initial aqueous solution (Step C). The active ingredients dissolve or otherwise diffuses from the quantity of plant matter into the quantity of distilled water. The waste solids are then filtered from the aqueous solution, in order to separate the active ingredients of the quantity of plant matter from undesirable solid matter (Step D). The aqueous solution is then lyophilized to form a lyophilized extract to concentrate suspended compounds of aqueous solution into a solid phase (Step E). The lyophilized extract is easier to transport and handle then the aqueous solution.

The active ingredients are then selectively separated from the lyophilized extract using the quantity of extracting agents into an active-ingredient extract (Step F), in order to separate the active ingredient from other compounds condensed into the lyophilized extract. The active-ingredient extract is subsequently eluted with the quantity of elution agents within the chromatography column to produce an impure product through thin layer chromatography (Step G). The impure product is then purified through chromatography within the chromatography column using the quantity of purification solvent to produce the antimalarial compound, 7-[(decahydroisoquinolin-6-yl) methyl] quinoxaline-2-carboxylic acid (Step H). Any remainder of the quantity of purification solvent mixed with the antimalarial compound is removed from the antimalarial compound in vacuo at −20° Celsius.

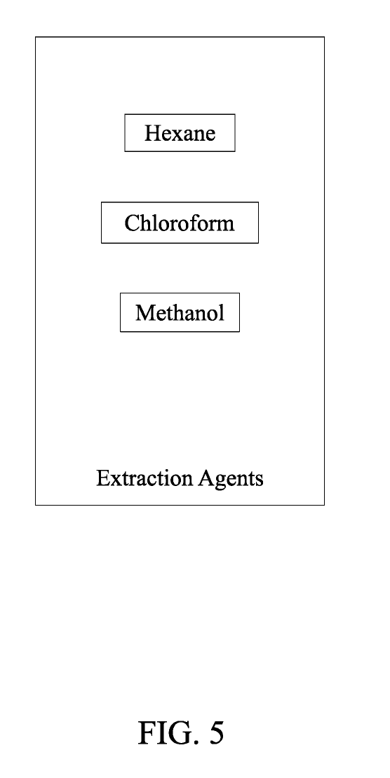

In accordance to FIG. 5, the quantity of extracting agent, from Step F, is preferred to be chloroform; however, the quantity of extracting agent is selected from the group consisting of a quantity of hexane, a quantity of chloroform, a quantity of methanol, and combinations thereof. Chloroform is the preferred compound for the quantity of extracting agent due to the selectivity for separating the active ingredient to provide a greater yield for the active-ingredient extract.

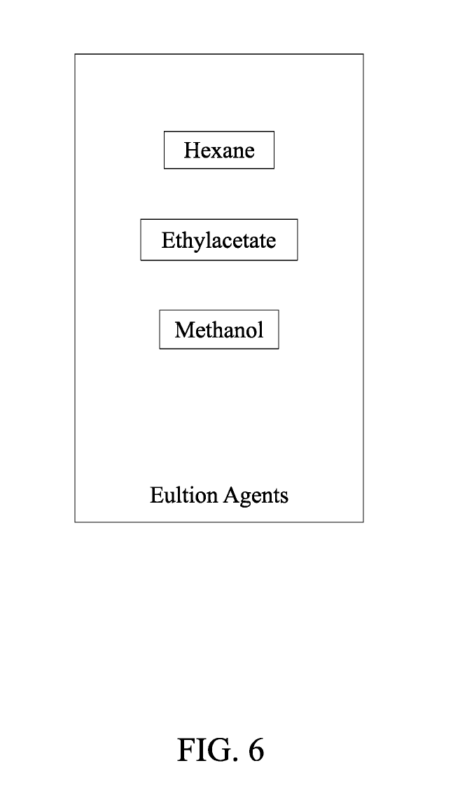

The quantity of elution agents, from Step G, is selected from a group consisting of a quantity of hexane, a quantity of ethylacetate, a quantity of methanol, and combinations thereof, shown in FIG. 6. The quantity of elution agents is preferred to be a combination of the quantity of ethylacetate and the quantity of methanol. The quantity of ethylacetate is preferred to be approximately 80% by volume (vol %) of the quantity of elution agents, and the quantity of methanol is preferred to be approximately 20 vol % of the quantity of elution agent. This composition is selected to provide the greatest yield for the impure product.

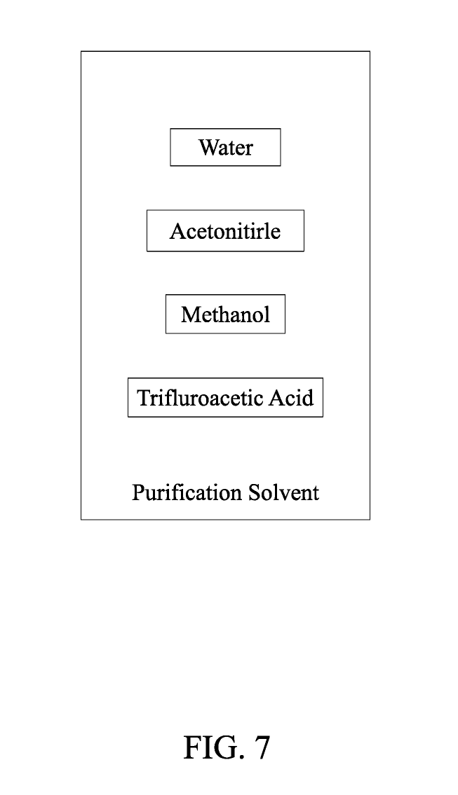

The quantity of purification solvent, from Step H, is selected from the group consisting of a quantity of acetonitirile, a quantity of methanol, a quantity of water, a quantity of trifluoroacetic acid, and combinations thereof, detailed in FIG. 7. The purification solvent separates the antimalarial compound from the remaining impurities of the impure product through high performance liquid chromatography. The purification solvent is preferred to be a combination of the quantity of acetonitrile, the quantity of methanol, the quantity of water, and the quantity of trifluoroacetic acid. The quantity of acetonitrile is preferred to be approximately 46 vol % of the quantity of purification solvent. The quantity of methanol is preferred to be approximately 15 vol % of the quantity of purification solvent. The quantity of water is preferred to be approximately 42 vol % of the quantity of purification solvent. The quantity of trifluoroacetic acid is preferred to be less than 1 vol % of the quantity of purification solvent. This composition separates the antimalarial compound from impurities of the impure product.

In accordance to some embodiments of the present invention, the antimalarial compound can be further synthesized to provide an improvement to the antimalarial activity and the safety profile over the antimalarial compound. Utilizing a quantity of synthesis agents, a derivative antimalarial compound is synthesized from the antimalarial compound and the quantity of synthesis agents.

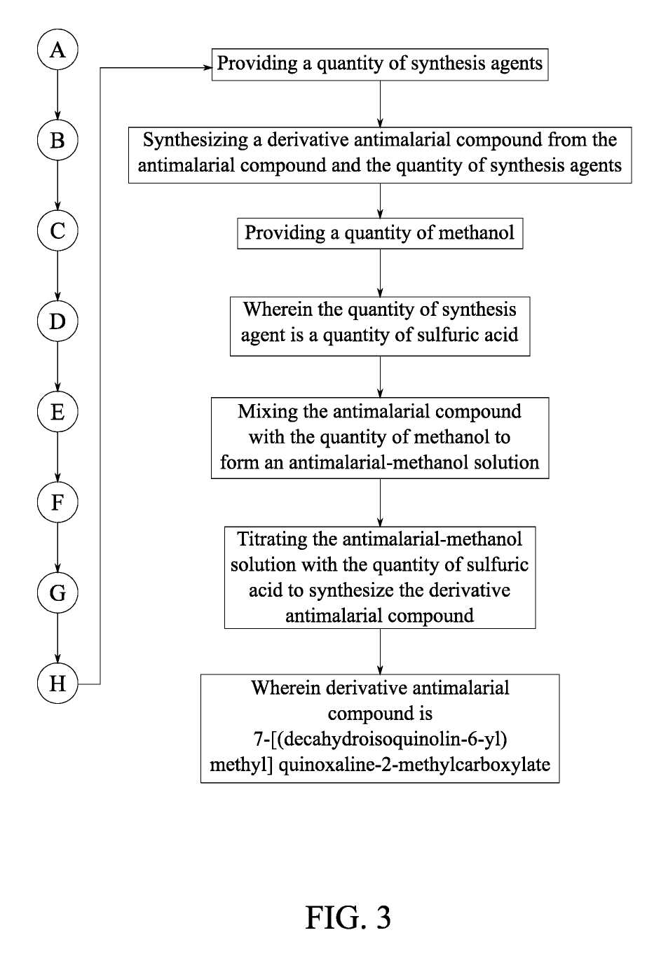

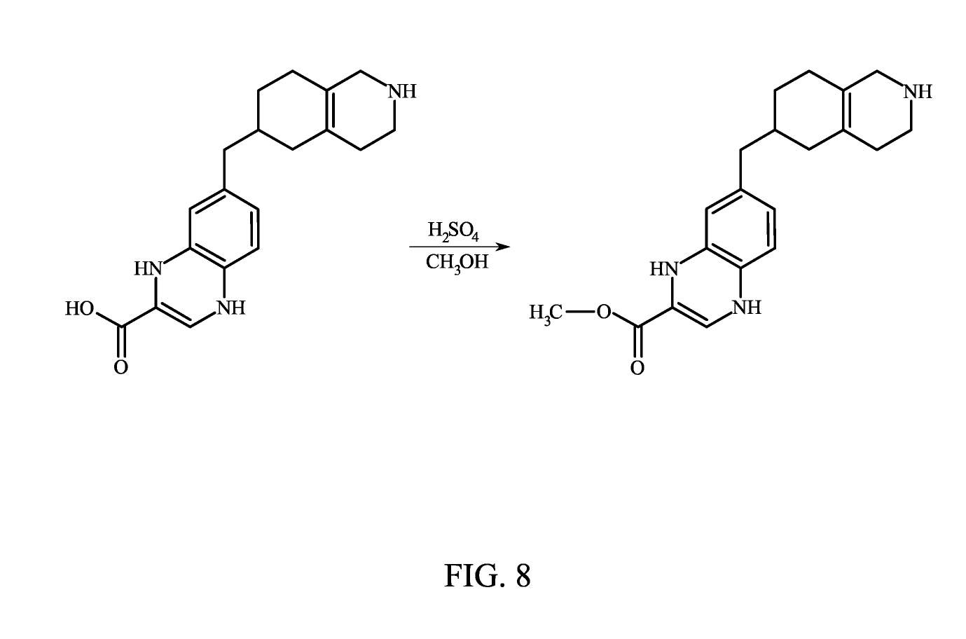

In a first alternate embodiment, the quantity of synthesis agents is a quantity of sulfuric acid, shown in FIG. 3. The antimalarial compound is mixed with a quantity of methanol to form an antimalarial-methanol solution. The antimalarial-methanol solution is titrated with the quantity of sulfuric acid to synthesize the derivative antimalarial compound, illustrated in FIG. 8. This embodiment of the present invention results in a derivative antimalarial compound that is 7-[(decahydroisoquinolin-6-yl) methyl] quinoxaline-2-methylcarboxylate.

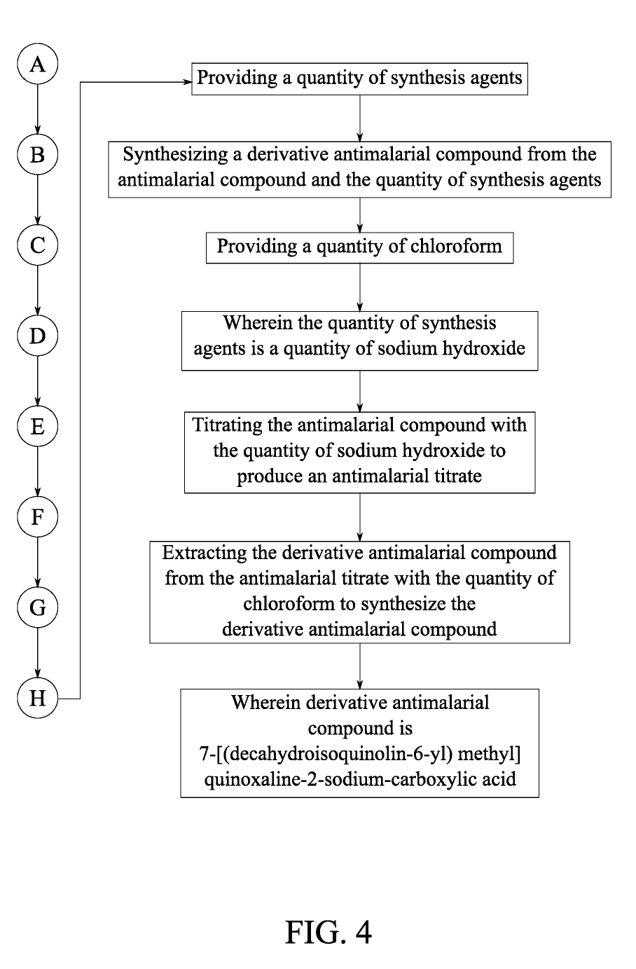

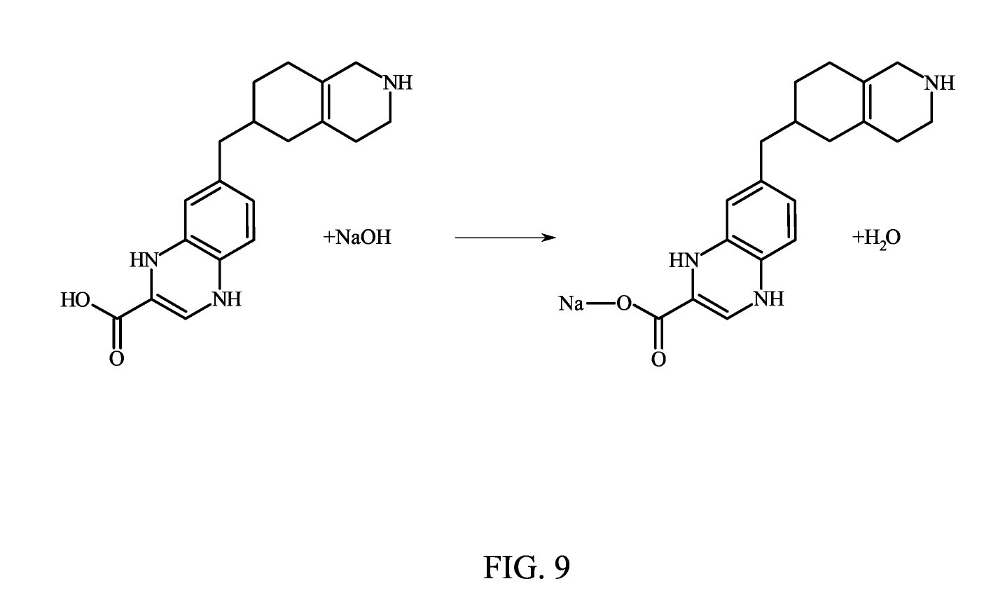

In a second alternate embodiment of the present invention, the quantity of synthesis agents is a quantity of sodium hydroxide, detailed in FIG. 4. The antimalarial compound is titrated with the quantity of sodium hydroxide to produce an antimalarial titrate to a pH between 10 and 11, illustrated in FIG. 9. The derivative antimalarial compound is extracted from the antimalarial titrate with the quantity of chloroform to synthesize the derivative antimalarial compound. This embodiment of the present invention results in a derivative antimalarial compound that is 7-[(decahydroisoquinolin-6-yl) methyl] quinoxaline-2-sodium-carboxylic acid.

Although the invention has been explained in relation to its preferred embodiment, it is to be understood that many other possible modifications and variations can be made without departing from the spirit and scope of the invention as hereinafter claimed.

What is claimed is:

1. A method for the production of an antimalarial compound comprises the steps of:
(A) providing a quantity of plant matter, a quantity of distilled water, a quantity of extraction agents, a quantity of elution agents, a quantity of purification solvent, and a chromatography column;
(B) reducing the particle size for the quantity of plant matter;
(C) submerging the quantity of plant matter into the quantity of distilled water to infuse active ingredients from the quantity of plant matter into the quantity of distilled water and form an initial aqueous solution;
(D) filtering waste solids from the aqueous solution;
(E) lyophilizing the aqueous solution to form a lyophilized extract;
(F) selectively separating the active ingredients from the lyophilized extract using the quantity of extracting agents into an active-ingredient extract;
(G) eluting the active-ingredient extract with the quantity of elution agents within the chromatography column to produce an impure product; and
(H) purifying the impure product through chromatography within the chromatography column using the quantity of purification solvent to produce the antimalarial compound.

2. The method for the production of an antimalarial compound, as claimed in claim 1, comprises the steps of:
providing a quantity of synthesis agents; and
synthesizing a derivative antimalarial compound from the antimalarial compound and the quantity of synthesis agents.

3. The method for the production of an antimalarial compound, as claimed in claim 2, comprises the steps of:
providing a quantity of methanol;
wherein the quantity of synthesis agent is a quantity of sulfuric acid;
mixing the antimalarial compound with the quantity of methanol to form an antimalarial-methanol solution; and
titrating the antimalarial-methanol solution with the quantity of sulfuric acid to synthesize the derivative antimalarial compound.

4. The method for the production of an antimalarial compound, as claimed in claim 3, wherein derivative antimalarial compound is 7-[(decahydroisoquinolin-6-yl) methyl] quinoxaline-2-methylcarboxylate.

5. The method for the production of an antimalarial compound, as claimed in claim 2, comprises the steps of:
providing a quantity of chloroform;
wherein the quantity of synthesis agents is a quantity of sodium hydroxide;
titrating the antimalarial compound with the quantity of sodium hydroxide to produce an antimalarial titrate; and
extracting the derivative antimalarial compound from the antimalarial titrate with the quantity of chloroform to synthesize the derivative antimalarial compound.

6. The method for the production of an antimalarial compound, as claimed in claim 3, wherein derivative antimalarial compound is 7-[(decahydroisoquinolin-6-yl) methyl] quinoxaline-2-sodium-carboxylic acid.

7. The method for the production of an antimalarial compound, as claimed in claim 1, wherein the quantity of plant matter is obtained from a plant of the species Cassia Nigricans.

8. The method for the production of an antimalarial compound, as claimed in claim 1, comprises:
the quantity of extraction agents being selected from the group consisting of a quantity of hexane, a quantity of chloroform, a quantity of methanol, and combinations thereof.

9. The method for the production of an antimalarial compound, as claimed in claim 1, comprises:
the quantity of elution agents being selected from the group consisting of a quantity of hexane, a quantity of ethylacetate, a quantity of methanol, and combinations thereof.

10. The method for the production of an antimalarial compound, as claimed in claim 9, comprises:
the quantity of elution agents being a combination of a quantity of ethylacetate and a quantity of methanol;
the quantity of ethylacetate being approximately 80% by volume (vol %) of the quantity of elution agents; and
the quantity of methanol being approximately 20 vol % of the quantity of elution agents.

11. The method for the production of an antimalarial compound, as claimed in claim 1, comprises:
the quantity of purification solvent being selected from the group consisting of a quantity of acetonitrile, a quantity of methanol, a quantity of water, a quantity of trifluoroacetic acid and combinations thereof.

12. The method for the production of an antimalarial compound, as claimed in claim 11, comprises:
the quantity of purification solvent being a combination of the quantity of acetonitrile, the quantity of methanol, the quantity of water, and the quantity of trifluoroacetic acid;
the quantity of acetonitrile being approximately 46 vol % of the quantity of purification solvent;
the quantity of methanol being approximately 15 vol % of the quantity of purification solvent;
the quantity of water being approximately 42 vol % of the quantity of purification solvent; and
the quantity of trifluoroacetic acid being less than 1 vol % of the quantity of purification solvent.

13. The method for the production of an antimalarial compound, as claimed in claim 1, wherein the antimalarial compound is 7-[(decahydroisoquinolin-6-yl) methyl] quinoxaline-2-carboxylic acid.
